(19) United States
(12) Patent Application Publication
(10) Pub. No.: US 2009/0036890 A1
(43) Pub. Date: Feb. 5, 2009

(54) FIXATOR APPARATUS WITH RADIOTRANSPARENT APERTURES FOR ORTHOPAEDIC APPLICATIONS

(76) Inventor: John Peter KARIDIS, Ossining, NY (US)

Correspondence Address: MYERS WOLIN, LLC, 100 HEADQUARTERS PLAZA, North Tower, 6th Floor, MORRISTOWN, NJ, 07960-6834, US

(21) Appl. No.: 12/183,054

(22) Filed: Jul. 30, 2008

Related U.S. Application Data
(60) Provisional application No. 60/962,620, filed on Jul. 31, 2007.

(51) Int. Cl. A61B 17/62

(52) U.S. Cl. 606/56

(57) ABSTRACT

An orthopaedic fixator apparatus with a first ring segment and a second ring segment for fixing a first and second bone element, a first post extending from the first ring segment towards the second ring segment, a second post extending from the second ring segment towards the first ring, a plurality of adjustable-length struts extending from the first ring segment and first post to the second ring segment and second post, wherein the lengths of the adjustable-length struts define the orientation of the first ring segment relative to the second ring segment, and wherein the apparatus provides a substantial central region free of x-ray obstruction. An additional embodiment enables convenient adjustment of the vertical compliance of the fixator by selectively disengaging one or more disengageable locking pins in one or more vertically oriented struts.

### CROSS REFERENCE TO RELATED APPLICATIONS

This application claims the benefit of U.S. Application 60/962,620, filed Jul. 31, 2007, the contents of which are incorporated herein by reference.

### FIELD OF THE INVENTION

This invention relates generally to the field of orthopaedic fixators and more specifically to a fixator providing large radiotransparent apertures positioned centrally during anterior-posterior and medial-lateral x-ray imaging.

### BACKGROUND

The Taylor Spatial Frame (http://www.jcharlestaylor.com/spat/00spat.html), which has the kinematic structure known to those skilled in the art of robotics as a “Stewart Platform”, or as a “Hexapod™”, provides full 6-degree-of-freedom control over the position and orientation of one bone segment relative to another bone segment.

Fixators are used to repair traumas or deformities, and a common post-operative requirement is the regular x-ray imaging of the bone to determine healing progress. An important deficiency of this structure is the x-ray obstruction caused by the numerous adjustable-length struts which extend at various angles from the lower ring or frame to the upper ring or frame. When viewed from the side, there are usually both open regions and obstructed regions near the central bone healing region. While some viewing directions may allow reasonably unobstructed views of the critical bone regions, it is very unlikely that both the medial-lateral view and the anterior-posterior view will be free of obstructions because there are 6 struts arranged in pairs at 120 degree intervals around the rings, while the normal x-ray imaging directions are 90 degrees apart.

### SUMMARY

An external fixator apparatus for orthopaedic application is disclosed having an arrangement of fixed-length or adjustable-length struts and rigid frames which substantially reduces the occlusion of x-ray images taken through two perpendicular imaging axes. In a preferred embodiment of the invention, upper and lower frame assemblies each comprise a full or partial support structure or ring section for attachment to a bone segment and a rigid extension structure or post protruding from the plane of each support structure or ring towards the other frame assembly, while preferably six fixed-length or adjustable-length struts, or a combination of the same, extending from the upper to the lower frame assembly define the relative position and orientation of the two frame assemblies in all six degrees-of-freedom. To minimize x-ray occlusion during imaging, the extension structures and the struts occupy regions substantially near or along the edges of a cube-like hexahedron, wherein the solid angle between any pair of adjacent fixed-length or adjustable-length struts is generally in the range of 45-135 degrees.

In another embodiment, a single preload ring and a single preload actuator are provided to preload the fixator structure, thus removing backlash in all joints and adjustable struts. In the illustrated embodiment, the preload ring is diagonally arranged to create a single preload force acting along a line passing near the centroid of the fixator, and can be constructed of radiolucent material, or shaped to avoid occluding the central region important for x-ray imaging if non-radiolucent, or simply removed for imaging.

In an alternative embodiment, a region of one or more struts includes alternating layers of rigid elements and elastic elements and at least one disengageable locking pin which prevent compression of an elastic element when engaged. The stiffness of the strut is adjusted by selectively engaging or disengaging one or more or the disengageable locking pins.

Other objects and advantages of the present invention will become apparent from the following descriptions, taken in connection with the accompanying drawings, wherein, by way of illustration and example, an embodiment of the present invention is disclosed.

### BRIEF DESCRIPTION OF THE DRAWINGS

The drawings constitute a part of this specification and include exemplary embodiments to the invention, which may be embodied in various forms. It is to be understood that in some instances various aspects of the invention are shown in simplified schematic form to facilitate an understanding of the invention.

FIG. 1 is a perspective view of the prior art.

FIGS. 2a and 2b are schematic diagrams of the prior art kinematic structure.

FIGS. 3a and 3b are schematic diagrams of a rectangular hexahedron structure with adjustable links, illustrating clear imaging axes through the structure.

FIGS. 4a and 4b are schematic diagrams illustrating that a rotated rectangular structure with adjustable links is kinematically equivalent to a Stewart platform.

FIGS. 5a-5d are schematic diagrams of alternative embodiments using rings or frames of different shape and extent.

FIGS. 6a-6c are schematic illustrations of an adjustable structure having semi-circular rings, in three orientations with different vertical heights, while FIGS. 6d-6g are schematic illustrations of an adjustable structure having semi-circular rings and formed primarily from fixed length struts.

FIG. 6h represents a section view of an adjustable-compliance region of a fixed-length or adjustable-length strut.

FIGS. 7a-7c contain perspective, front and side views of an alternative embodiment, illustrating the clear central imaging regions.

FIGS. 8a and 8b illustrate the diagonal preloading of the invention.

FIG. 9 is a perspective view with diagonal preloading means.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

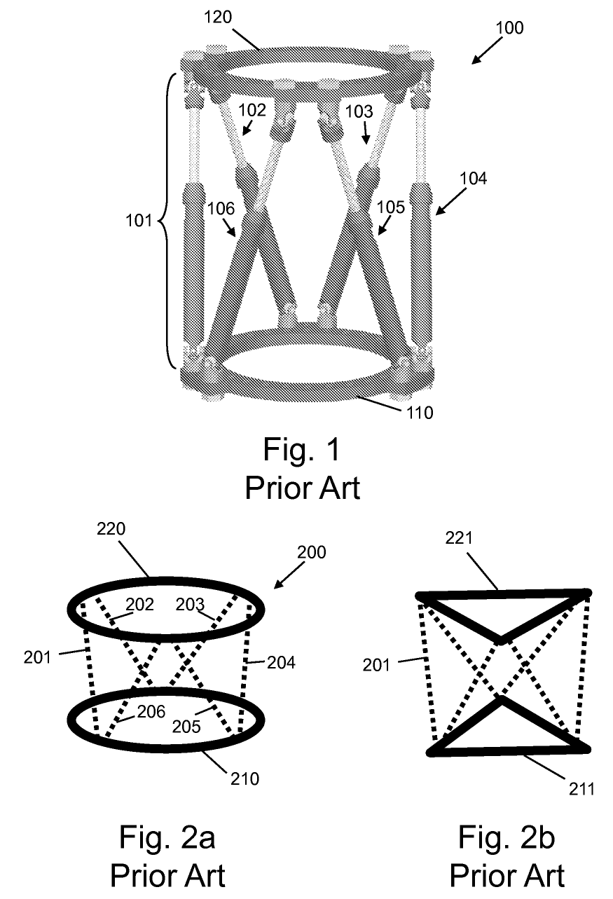

FIG. 1 shows a perspective view of the prior art Taylor Spatial Frame fixator 100 from Smith & Nephew. This fixator 100 has the kinematic structure sometimes known as a “Stewart Platform”, and comprises a lower ring 110 attachable to a first bone segment (not shown), an upper ring 120 attachable to a second bone segment (not shown), and a plurality of adjustable-length struts 101-106 with multi-pivoting end-joints. When these adjustable length struts are arrayed in a particular alternating pattern between three mounting regions on the lower ring 110 and three mounting regions on the upper ring 120, the length of the adjustable length struts 101-106 defines the position and orientation of the upper ring 120 relative to the lower ring 110, in all six possible degrees-of-freedom (DOF) comprising three translations and three rotations.

FIG. 2a schematically illustrates the kinematic structure 200 of the Taylor Spatial Frame, with dotted lines representing adjustable length struts 201-206 and with heavy solid lines representing rigid rings 210 and 220. In this and subsequent figures, each dotted line is used to represent an entire adjustable length strut, complete with pivoting end-joints, such that each dotted line is kinematically equivalent to an adjustable length strut with pivoting end-joints such as 101 from FIG. 1. FIG. 2b illustrates a completely equivalent kinematic structure where rigid circular rings 210 and 220 have been replaced by rigid triangular rings 211 and 221. While prior art implementations generally use circular rings, this kinematically equivalent structure with triangular rings will be used to illustrate the relationship between the prior art and the disclosed embodiments.

While the prior art construction provides the desired six-DOF control, it has a significant deficiency in that the angled adjustable struts often block important regions of diagnostic x-rays taken of patients wearing the frame. While there may be a clear region for images passing through the centroid of the overall apparatus, the region is relatively small, and the 120-degree spacing of the struts around the rings makes it very likely that a clear region in one imaging plane will be an obstructed region in an orthogonal imaging plane. Therefore, it is an important aspect of the disclosed embodiments to provide a six-DOF adjustable fixator assembly which provides a relatively larger unobstructed view through the centroid of the frame for two orthogonal imaging directions.

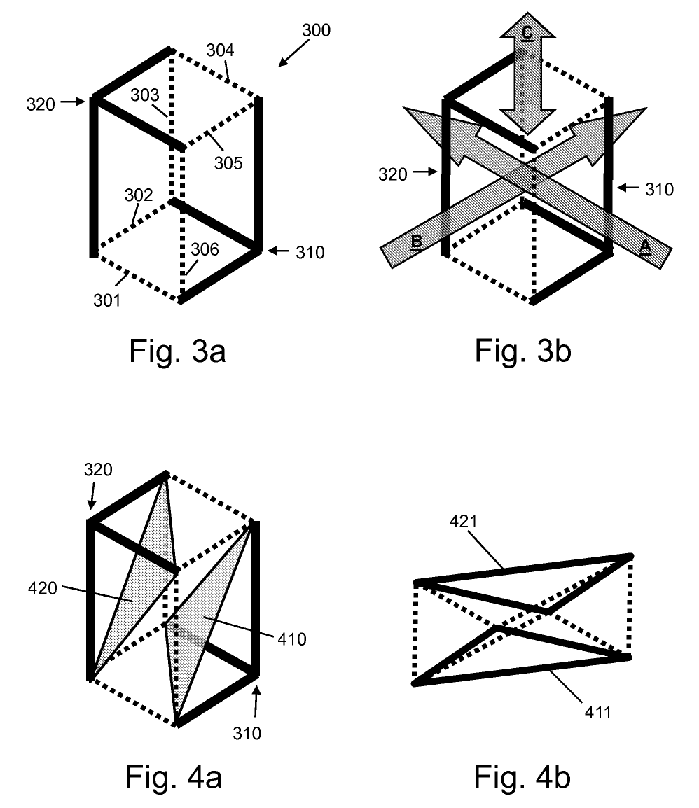

FIG. 3a is a schematic representation of one embodiment of the present invention, which in this case is an open cube-like structure 300 with six adjustable length struts, 301-306, attached between the ends of a lower rigid tripod element 310 and the ends of an upper rigid tripod element 320. FIG. 3b illustrates one clear imaging axis A and a second orthogonal clear imaging axis B, both of which are orthogonal to the typical bone or limb axis, C. It will be clear to those skilled in the art that the rigid tripod elements 310 and 320 may have substantially different shapes without departing from the essential objective of the structure, which is to provide three strut attachment regions or points which are displaced distally from a common rigid joining point. In a preferred embodiment, the common rigid joining point lies outside of an unobstructed cylindrical region which provides clearance for the patient's limb. Furthermore, in a preferred embodiment where an identical or a similar rigid structure is employed for both frames, the two common rigid joining points for the two rigid structures are naturally located at diagonally opposite corners of a rectangular or cube-like hexahedron.

To more clearly illustrate the kinematic equivalence between the adjustable cube-like structure of FIG. 3b and a Stewart Platform, FIG. 4a shows the structure from FIG. 3a with a rigid triangular plate 410 attached to the ends of the legs from rigid tripod element 310, and a second rigid triangular plate 420 attached to the ends of the legs from rigid tripod element 320. The addition of the rigid triangular plates to a rigid tripod element does nothing to change the kinematics of the structure. Furthermore, the subsequent removal of the rigid tripod elements after the rigid triangular plate is added does nothing to change the kinematics of the structure.

FIG. 4b shows a kinematically equivalent structure where rigid triangular plates 410 and 420 have been replaced by rigid open triangular frames 411 and 421. The entire structure has also been rotated to more closely match the orientation of the structure shown in FIG. 2b. It will now be clear to those skilled in the art that the structure in FIG. 4b is kinematically equivalent to the Stewart platform with triangular frames illustrated in FIG. 2b. Since FIG. 4b is kinematically equivalent to the cube-like schematic representation of this invention as shown in FIG. 3a, it has been clearly shown that the current invention is kinematically equivalent to, and has the same six-DOF adjustment capability as a Stewart platform used in the prior art. However, the present invention provides significantly less obstruction along imaging axes A and B (FIG. 3b). This improved imaging capability is a surprising result of the positioning of the adjustable length struts along the edges of a cube-like hexahedron structure and the three-dimensional (non-planar) nature of the tripod-like corner structures.

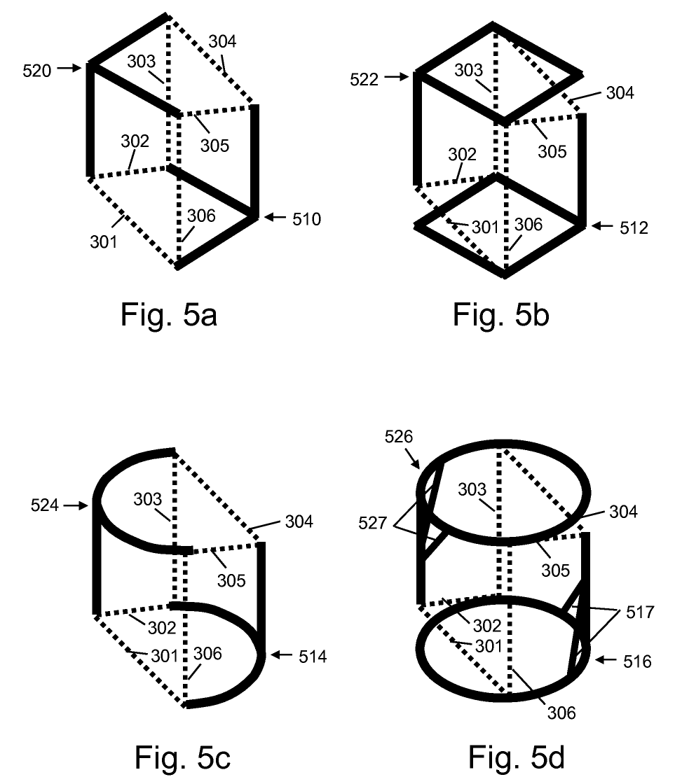

FIG. 5a illustrates the same basic structure shown in FIG. 3a, but the vertical legs on tripod elements 510 and 520 have been shortened somewhat from those on elements 310 and 320. Since the vertical separation between the corners of the frames was held constant, the adjustable links 301, 302, 304, and 305 are no longer exactly aligned with the edges of a cube-like hexahedron.

FIG. 5b illustrates the same kinematic structure as shown in FIG. 5a, but in this embodiment the lower rigid tripod element 510 has been replaced with a square ring and post structure 512. Similarly, the upper rigid tripod element 520 has been replaced with a square ring and post structure 522. The complete square ring portions of 512 and 522 provide additional stiffness as well as more flexibility for the orthopaedic surgeon who must use various wires or pins to attach a bone element to the ring structures. It will be clear to those skilled in the art that the frame created by portions of the tripod element can take on any appropriate shape.

FIG. 5c shows an alternative embodiment where the square frame portions of square ring and post structures 512 and 522 of FIG. 5b have been replaced by semi-circular ring and post structures 514 and 524.

FIG. 5d illustrates another embodiment where the semi-circular ring and post structures 514 and 524 in FIG. 5c have been replaced by full ring and post structures 516 and 526. It will be clear that the shape of the post extension is not limited to the simple cantilevered post shown in FIGS. 5a-5c. As an example, FIG. 5d illustrates the addition of optional stiffeners 517 and 527 which improve the strength and stiffness of ring and post structures 516 and 526. It will also be clear that other structures are possible without deviating from the scope or intent of this invention. Therefore, the term “post” in this disclosure is generally intended to mean any rigid extension from a full or partial ring or support structure, protruding generally towards the other full or partial ring or support structure, in any shape that provides a relatively rigid mounting point displaced from the ring structure, while also minimizing x-ray imaging obstruction by any radio-opaque structural elements.

Another significant advantage of the disclosed embodiments is that for small position adjustments around a nominally rectangular hexahedron-shaped starting position, the required changes in adjustable strut lengths can be determined intuitively, whereas calculating the strut length adjustments needed to create a given positional change in the Taylor Spatial Frame of the prior art, for example, is so complex as to almost always require computer assistance.

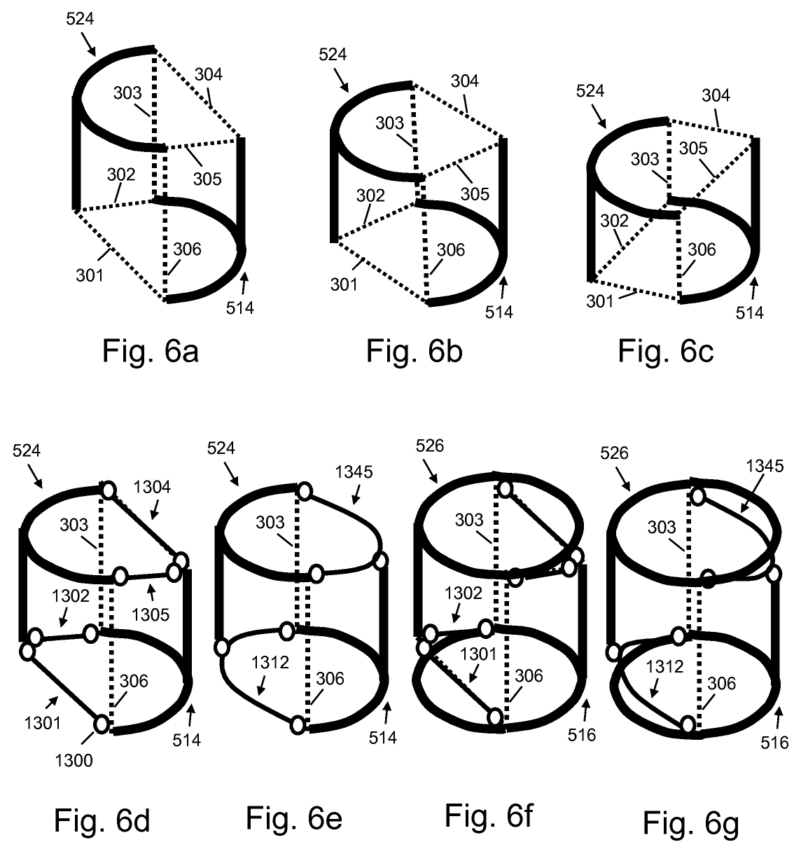

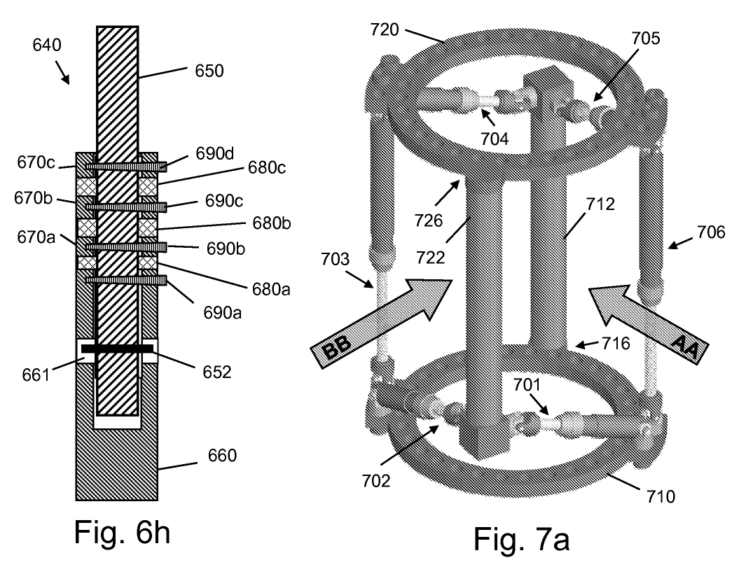

This is illustrated in FIGS. 6a-6c that show one embodiment of the invention in three different positions, where only the length of adjustable length struts 303 and 305 have been changed. As will be appreciated by those skilled in the art, the structure shown in FIGS. 6a-6c has the following useful translational properties:

Vertical relative translation of the two frame structures 514 and 524 is controlled primarily by making equal changes to adjustable length struts 303 and 306.

Horizontal relative translation of the two frame structures 514, 524 in one direction is controlled primarily by making equal changes to adjustable length struts 301 and 304.

Horizontal translation in the orthogonal direction is controlled primarily by making equal changes to adjustable length struts 302 and 305.

The relative rotation of the two frame structures 514, 524 can be controlled by making equal magnitude but opposite sign adjustments to selected struts, and the structure shown in FIGS. 6a-6c also has the following useful rotational adjustment properties.

Axial relative rotation of the two frame structures 514, 524 is controlled primarily by making equal changes to adjustable length struts 302 and 304, while making equal magnitude but opposite sign (i.e., lengthening instead of shortening, or vice-versa) changes to adjustable length struts 301 and 305.

Relative tilt adjustment of the two frames structures 514, 524 around one axis is controlled primarily by making equal but opposite changes to adjustable length struts 301 and 304.

Relative tilt adjustment of the two frame structures 514, 524 around the orthogonal axis is controlled primarily by making equal and opposite changes to adjustable length struts 302 and 305.

While the embodiments illustrated in FIGS. 5a-d and FIGS. 6a-c contain six adjustable struts, the scope of the invention is not limited to requiring the use of six adjustable length struts as illustrated, and if fewer than six degrees of freedom of adjustability are required. As a non-limiting example, FIG. 6d schematically illustrates an embodiment using four fixed length struts 1301, 1302, 1304, 1305 in place of adjustable length struts 301, 302, 304, 305 in FIG. 6a. In the schematic representations of FIGS. 6d-6g, pivoting joints at the ends of the fixed length struts are illustrated as open circles, and each fixed length strut comprises both a rigid portion illustrated by a medium-weight solid line, plus two or more pivoting joints illustrated with the open circles. Because the structure illustrated in FIG. 6d has only two adjustable length struts (303 and 306) the fixator can only have two adjustable degrees of freedom, in this case comprising primarily vertical height plus one axis of combined translation and rotation. It should be appreciated that these various embodiments are not meant to be limiting in any sense, but are described for purposes of illustration and remain consistent with the advantage of providing a multi-DOF fixator that is easily adjustable and with improved imaging capabilities.

FIG. 6e illustrates another embodiment wherein fixed length struts 1301 and 1302 are combined into a single fixed-length curved strut 1312. Similarly, fixed length struts 1304 and 1305 are combined into a single fixed-length curved strut 1345. In this embodiment, the adjustable length struts 303 and 306 provide primarily vertical adjustability and should be adjusted equally, as the rigid nature of the combined links 1312 and 1345 will resist adjustments made with unequal length adjustments of struts 303 and 306. FIGS. 6f and 6g illustrate the same kinematic structures shown in FIGS. 6d and 6e, wherein the semicircular ring and post structures 514 and 524 have been replaced by full ring and post structures 516 and 526.

One of the important advantages that results from these characteristics is that the stiffness of the structure in the vertical direction is almost completely determined by the stiffness of adjustable length struts 303 and 306. After many orthopaedic procedures, the surgeon would like to be able to reduce the axial stiffness of the frame prior to its complete removal, so that the repaired bone joint can be axially loaded with a larger fraction of any externally applied loads from daily activities or exercises. This procedure can help to ensure that the bone is fully healed and capable of withstanding external loads before the frame is removed, and it can substantially reduce the occurrence of re-fracture (and additional surgery) after frame removal.

When using prior art orthopaedic fixators such as the Taylor Spatial Frame, for example, the stiffness of the adjustable frame is dependent on the stiffness of many strut elements in a complex and non-obvious way. Removing one strut, as is sometimes done, eliminates the constraint on one degree of freedom, and the frame is free to rotate and twist in unintended directions. Controllably reducing the stiffness in the axial direction would require a stiffness change in most or all adjustable strut elements. By contrast, using an embodiment as discussed herein, the vertical (or axial) stiffness of the frame can be reduced by reducing the stiffness of the two mostly vertical adjustable length struts 303 and 306. Such a reduction in stiffness can be achieved by the surgeon either by replacing the original vertical struts 303 and 306 with equivalent-length struts having lower stiffness, or through the use of adjustable length struts which can also be adjusted to have a different stiffness.

FIG. 6h provides a cross-section view of a compliance adjustment feature that can be built into any fixed-length or adjustable-length strut disclosed herein. The adjustable compliance strut region 640 is comprised of a first rigid strut element 650, a second rigid strut element 660, an alternating stack of small rigid elements 670a-c and small compliant elements 680a-c, together with one or more disengageable locking pins 690a-d. When locking pin 690a is engaged, the strut incorporating such strut region 640 has maximum stiffness because pin 690a prevents relative motion of strut elements 650 and 660. If pin 690a is removed or otherwise disengaged, but pin 690b remains engaged, forces acting between strut elements 650 and 660 can cause compression (or extension) of compliant element 680a, thus resulting in a desired decrease in strut stiffness. Furthermore, subsequent removal or other disengagement of pins 680b and 680c will result in further reductions in strut stiffness as compliant elements 680b and 680c can now be compressed (or extended). Lastly, removal or other disengagement of pin 690d would allow free relative motion of strut elements 650 and 660, up to the limits defined by the clearance between an optional limit pin 652 attached to strut element 650 and situated within a limit cavity 661 in strut element 660. In this manner, the stiffness of the strut incorporating such strut region 640 is determined by the number and stiffness of the compliant regions which are not locked into place. The surgeon can reduce the stiffness of the strut by simply disengaging one or more locking pins. It will be clear to those skilled in the art that locking pins 690a-d can be cylindrical, tapered, or having localized flats or other non-round cross-sectional shapes or the like, and that disengagement can be achieved by complete pin removal, partial pin removal, rotation of a non-round pin, or other means, and that the limit stop function created by limit pin 652 and limit slot 661 can also be achieved by many alternate means.

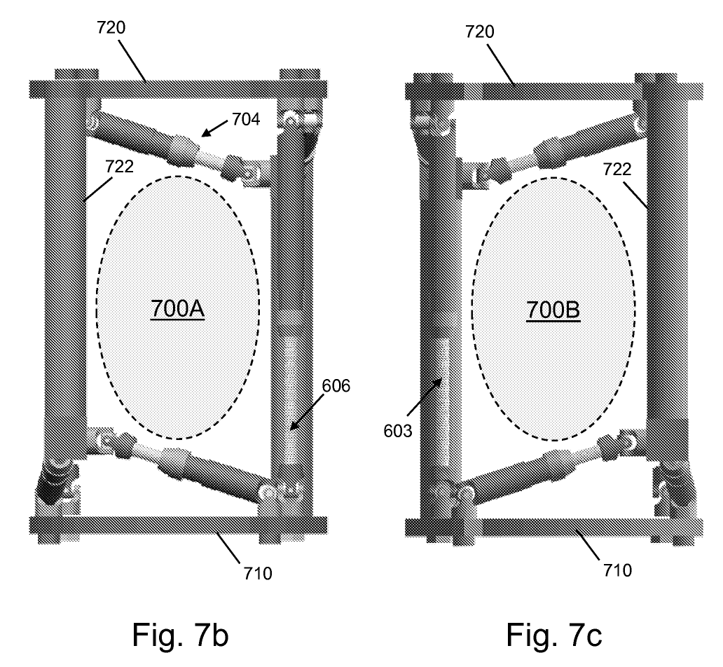

FIG. 7a shows a perspective view of a preferred embodiment corresponding to the schematic illustration in FIG. 5d. As can be seen, the fixator embodiment in FIG. 7a has a lower circular ring 710 or support with a rigidly attached post 712 extending upwards, and an upper circular ring 720 or support with a rigidly attached post 722 extending downwards. The posts 712 and 722 do not reach all the way to the plane of the opposite ring and are effectively spaced therefrom along the longitudinal axis of the posts to avoid interference with the rings themselves, but they do extend a substantial fraction of the distance to the other ring in order to keep the diagonal strut elements 701, 702, 704 and 705 from interfering with imaging in the centroid region of the fixator. The combination of circular ring 710 and extension post 712 forms a lower ring and post structure 716, which is analogous to the rigid ring and post structure 516 in FIG. 5d. Similarly, the combination of circular ring 720 and extension post 722 forms an upper ring and post structure 726 which is analogous to 526. Reinforcement struts 517 and 527 in FIG. 5d can be optionally added to ring and post structures 716 and 726 if additional structural stiffness is desired. In the illustrated embodiment, six adjustable length struts 701-706 extend in an alternating pattern from the lower ring and post structure 716 to the upper ring and post structure 726. Thus, the structure shown in FIG. 7a represents a six-DOF fixator satisfying the previously unattainable goal of allowing unobstructed x-ray imaging of the region near the centroid of the frame, from imaging axis AA (FIG. 7b) and orthogonal imaging axis BB (FIG. 7c).

FIGS. 7b and 7c show typical anterior-posterior and medial-lateral views respectively of the fixator as it would be positioned for typical x-rays of the bone and tissue being stabilized (not shown) by the fixator. The very large and totally unobstructed regions encompassing the regions 700A and 700B near the centroid of the frame are clearly shown. It will be obvious to those skilled in the art that other variations of this structure are possible, and the angled adjustable struts can be moved even further away from the central region if only limited further adjustment is required, or if portions of the rings are removed as was illustrated in other embodiments described herein including, but not limited to the embodiment of FIG. 5c for example. Similarly, straight struts can be replaced with curved struts if additional clearance is desired. Other configurations are possible.

One minor disadvantage of the fixator structure shown in FIGS. 5a-5d and FIGS. 7a-7c, for example, is that adjustment of the struts to produce extreme translation or rotation of one ring relative to the other can produce interference between the rigid post and the opposite ring or the patient's limb, or can produce a structure where the rigid post extends away from the frame centroid to an undesirable degree. However, the majority of applications for external fixators are for trauma repair or reconstructive surgery where the upper and lower rings do not take extreme relative positions, but instead maintain centers that are reasonably aligned above one another, and with reasonably small relative tilt angles. For these applications, the slightly reduced practical range of adjustment is not a significant disadvantage, while the improved x-ray imaging capability, and the optional ability to adjust compliance with two vertical struts, represent significant advantages.

One potential deficiency of virtually all fixators controlled by adjustable length struts is that unavoidable manufacturing clearances and tolerances result in some amount of free play or “backlash” in the system, which prevents the structure from precisely and rigidly holding one ring or frame (and attached bone segment) relative to the other ring or frame (and attached bone segment). One method for reducing or eliminating the deleterious effects of backlash includes the use of multiple additional preload actuators which are arranged to provide preloading of all joints in all six adjustable struts. The current invention can also be preloaded to reduce backlash in a similar manner, but one non-limiting embodiment disclosed herein has the additional benefit of being able to be fully preloaded using only one preload actuator.

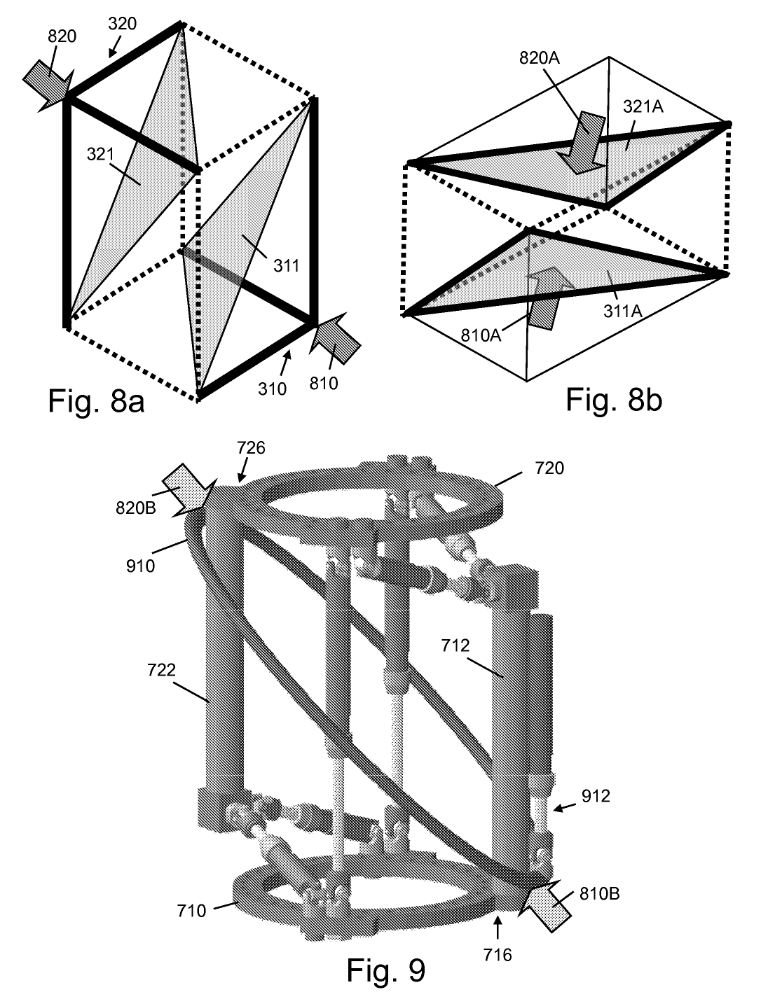

The operation and efficacy of a single preload actuator is illustrated schematically in FIGS. 8a and 8b, which illustrate how a single set of forces 810 and 820 acting diagonally from one rigid corner to the opposite rigid corners in FIG. 8a is equivalent to a set of axial preload forces 810A and 820A on the equivalent Stewart platform shown in FIG. 8b. It will be clear to those skilled in the art that a single preload force acting along a line passing near the centroid of the structure in FIG. 8b will preload all joints of all six adjustable struts. Thus, when rotated to the orientation of FIG. 8a, a single preload force acting across the diagonally opposed rigid corners will also effectively preload all joints of all adjustable struts.

FIG. 9 shows a perspective view of one embodiment of the fixator previously illustrated in FIG. 7a, with the addition of an elliptical ring 910 extending from one diagonal corner of ring and post structure 726 to the diagonally opposite corner of ring and post structure 716, together with a preload actuator 912 supported by the ring and post structure 716, which can be adjusted to force one end of the ring 910 closer to the origin of extension post 712 in such a manner that the resulting forces 810B and 820B pull diagonally opposed rigid ring and frame elements 716 and 726 towards each other. The elliptical ring can be very rigid or it can be somewhat compliant, for example, and it can be radiolucent or radio-opaque, all without deviating from its preload functionality. If the elliptical ring 910 is radio-opaque, it can be removed temporarily during x-ray imaging to avoid occluding the resulting images, without affecting the kinematic stability or basic positioning of the frame. While the ring 910 is shown with a particular shape and in a particular configuration relative to the frame elements 716, 726, it will be understood that other positioning and configurations of the ring 910 and/or actuator 912 relative to the fixator are contemplated. For example, the ring 910 might extend from positions along the lengths of the posts 712, 722 as the case may be. Other configurations are contemplated.

Thus, the fixator of the illustrated embodiments provides full six-DOF positioning control, if desired, which preferably does not occlude or substantially occlude the important central region during x-ray imaging from the two typical orthogonal directions. There is also provided the ability for placement of a single preload actuator for removing the backlash caused by all joints of all adjustable struts.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

1. A fixator for orthopaedic applications comprising:
a) a first ring segment for directly or indirectly fixing a first bone element;
b) a second ring segment for directly or indirectly fixing a second bone element;
c) a first rigid post extending from the first ring segment towards the second ring segment;
d) a second rigid post extending from the second ring segment towards the first ring segment;
e) a plurality of fixed or adjustable length struts extending from an assembly of the first ring segment and first rigid post to an assembly of the second ring segment and second rigid post;
f) wherein the length of the fixed or adjustable length struts define the position and orientation of the first ring segment relative to the second ring segment in one or more degrees of freedom, and wherein the arrangement of all elements produces a central region that is substantially free of x-ray obstruction when imaged in two orthogonal directions.

2. The fixator of claim 1, wherein said two orthogonal directions are generally perpendicular to an axis passing through the centers of the first and second ring segments.

3. The fixator of claim 1, wherein said first ring segment has the general form of one of: a full circle, a partial circle, an ellipse, an arc, a square, a portion of a square, or other polygonal shape.

4. The fixator of claim 1, wherein said plurality of fixed or adjustable length struts further comprises:
a) a first adjustable strut extending from the first ring segment to the second ring segment in a direction generally parallel to a central axis generally defined between the centers of the first and second ring segments;
b) a second adjustable strut extending from the first ring segment to the second ring segment in a direction generally parallel to the central axis;
c) two adjustable third struts extending from the first rigid post to the second ring segment; and
d) two adjustable fourth struts extending from the second rigid post to the first ring segment.

5. The fixator of claim 4, wherein the angle between any two adjacent adjustable struts is between 45 and 135 degrees.

6. A fixator for orthopaedic applications comprising:
a) a first rigid frame segment with a first three attachment regions positioned distally from a first joining point;
b) a second rigid frame segment with a second three attachment regions positioned distally from a second joining point;
c) six fixed-length or adjustable length struts extending from the three first attachment regions on the first rigid frame segment to the second three attachment regions on the second rigid frame; wherein
d) the first joining point and the second joining point are outside of an open cylindrical region in the center of the fixator, and wherein
e) the solid angle between any pair of lines extending from the first joining point to the first three attachment regions is in the range of 45 to 135 degrees.

7. The fixator of claim 6, wherein the first and second joining points are defined at diagonally opposite corners of the fixator.

8. A fixator for orthopaedic applications comprising:
a) a first support for directly or indirectly fixing a first bone element;
b) a second support for directly or indirectly fixing a second bone element;
c) a plurality of height-defining struts connected between the first and second supports that define the height of the fixator;
d) a plurality of fixed-length posts, each having a first end connected to the first or second support, and a second end spaced from the other support along a longitudinal axis of the post; and
e) a plurality of connecting struts connecting the second end of each post to the support from which the second end is spaced;
f) the arrangement of the struts and posts producing a central region that is substantially free of x-ray obstruction when the fixator is imaged in two orthogonal directions.

9. The fixator of claim 8, wherein the height-defining struts are adjustable length struts.

10. The fixator of claim 9, further comprising a pair of connecting struts associated with each second end, each connecting strut extending between its respective second end and the support from which the second end is spaced, along an axis that is not parallel to the longitudinal axis of the post associated with such second end.

11. The fixator of claim 8, further comprising a preload element extending around at least one of the fixed-length posts and the height-defining struts for reducing backlash.

12. The fixator of claim 11, further comprising an elliptical ring extending diagonally from one corner of the fixator to another corner for creating a single preload force acting along a line passing near the centroid of the fixator.

13. The fixator of claim 12, further comprising an adjustable preload actuator for adjusting a force of the preload element.

14. The fixator of claim 8, wherein one of the struts further comprises a first strut element, a second strut element, and at least one compliant element which is compressed when the first strut element is moved towards the second strut element.

15. The fixator of claim 14, wherein the one of the struts further comprises at least one disengageable locking pin that substantially prevents compression of the at least one compliant element when the disengageable locking pin is engaged.

16. The fixator of claim 15, wherein the at least one compliant element comprises an alternating stack of rigid and compliant sub-elements.

17. The fixator of claim 16, wherein sequential removal of the at least one disengageable locking pins produces a sequential reduction in the stiffness of the one of the struts.

18. A fixator for orthopaedic applications comprising:
a) a first support for directly or indirectly fixing a first bone element;
b) a second support for directly or indirectly fixing a second bone element;
c) a plurality of struts connected between the first and second supports that are arranged to produce a substantial central region free of x-ray obstruction when the fixator is imaged in two orthogonal directions; and
d) a preload element for creating a single preload force acting along a line passing near the centroid of the fixator.

19. The fixator of claim 18, wherein the preload element extends around the plurality of struts.

20. The fixator of claim 19, wherein the preload element further comprises an elliptical ring extending diagonally from one corner of the fixator to another corner.

21. The fixator of claim 20, further comprising an adjustable preload actuator for adjusting the force of the preload element.

22. (canceled)

23. (canceled)

24. (canceled)

25. (canceled)

26. (canceled)

27. (canceled)

28. (canceled)
